(19) United States
(12) Patent Application Publication
(10) Pub. No.: US 2016/0130652 A1
(43) Pub. Date: May 12, 2016

(54) USE OF URINARY HIV NUCLEIC ACIDS TO DIAGNOSE AND MONITOR HIV-ASSOCIATED NEPHROPATHY (HIVAN)

(71) Applicants: INSERM INSTITUT NATIONAL DE LA SANTÉ ET DE LA RECHERCHE MÉDICALE, Paris (FR); UNIVERSITE PARIS DESCARTES, Paris (FR); ASSISTANCE PUBLIQUE-HOPITAUX DE PARIS (APHP), Paris (FR)

(72) Inventors: Guillaume CANAUD, Paris (FR); Fabiola TERZI, Paris (FR); Christine ROUZIOUX, Paris (FR)

(21) Appl. No.: 14/894,035

(22) PCT Filed: May 28, 2014
(86) PCT No.: PCT/EP2014/061108

§ 371 Date: Nov. 25, 2015

(30) Foreign Application Priority Data
May 28, 2013 (EP) 13305697.8

(51) Int. Cl. C12Q 1/68; C12Q 1/70

(52) U.S. Cl. CPC C12Q 1/6883; C12Q 1/703; C12Q 2600/158

(57) ABSTRACT

The invention relates to the diagnosis of HIV-associated nephropathy (HIVAN) based on determining the expression level of HIV nucleic acids (HIV RNA and/or DNA) in combination with the renal function of a patient. The invention also relates to a method for distinguishing between HIVAN and non-HIVAN kidney disease in a patient based on the expression level of urinary HIV nucleic acids.

### FIELD OF THE INVENTION

The invention provides methods and kits for diagnosing HIV-associated nephropathy (HIVAN) based on determining the expression level of HIV nucleic acids in a urine sample obtained from a patient in combination with the renal function of said patient. The invention also relates to a method for distinguishing between HIVAN and non-HIVAN kidney disease in a patient based on the expression level of urinary HIV nucleic acids.

### BACKGROUND OF THE INVENTION

More than thirty years after the first description of Acquired Immunodeficiency Syndrome (AIDS), kidney disease remains an important contributor to morbidity and mortality in HIV infected patients {Adih et al., 2011; Wyatt et al., 2012}. The introduction of highly active antiretroviral therapy (HAART) fifteen years ago has completely modified the prognosis of HIV-infected patients, significantly reducing mortality and increasing life expectancy {Cohen et al., 2011}. However, end stage renal disease (ESRD) in this population remains common {Wyatt et al., 2012}. Indeed, HIV represents the third cause of ESRD among African Americans under the age of 65 years, and approximately 900 HIV infected patients per year go onto dialysis in the United States {Wyatt et al., 2012}.

Kidney lesions during HIV infection are mainly due to a particularly aggressive form of focal and segmental sclerosis (FSGS) called HIV-associated nephropathy (HIVAN). This disease is directly related to infection of kidney cells by HIV {Wyatt et al., 2012; Marras et al., 2002}. Until recently, long-term dialysis was the only treatment available in this group of patients and was associated with a poor prognosis. Since recent reports showing favorable outcomes, kidney transplantation has become a therapeutic option for these patients {Stock et al., 2010 #4363; Kumar et al., 2005; Touzot et al., 2010}. Most centers have defined eligibility criteria for transplantation such as a CD4 count >200 cells/mL and undetectable plasma HIV RNA {Am J Transplant, 2004}. Using these criteria, HIV infected transplant recipient survival rates are similar to that of non-HIV transplant recipients, but intriguingly three-year allograft survival rate is reduced {Stock et al., 2010}. Mechanisms that lead to this shortened allograft survival rate are not known and at least two explanations are usually suggested {Stock et al., 2010}. The first one is the impact of the poorly understood high rate of acute cellular rejection observed during the first months after transplantation. The second one is the higher exposure to calcineurin inhibitor blood levels due to pharmacokinetic interactions with some antiretroviral drugs. Nevertheless, these explanations are not completely satisfying and very little attention has been devoted to the potential infection of the kidney allograft by HIV.

However, identifying the involved mechanisms would be very useful for an early identification of HIVAN. Such early diagnosis is important because HAART, corticosteroids, and inhibition of renin-angiotensin may delay disease progression. Nonetheless, because HIV infection may be associated with other glomerular disease, definitive diagnosis of HIVAN requires a kidney biopsy. Therefore, there is a great need for new biomarkers of HIVAN that would be useful for reliable diagnosing from the early steps of the disease (e.g. early FSGS lesions) but also for determining whether a human patient is a candidate for treatment with HAART, monitoring the treatment as well as the progression of HIVAN in treated patients.

### SUMMARY OF THE INVENTION

In a first aspect, the invention relates to a method for diagnosing HIV-associated nephropathy (HIVAN) in a HIV-infected patient, comprising the following steps of:

a) determining the expression level of HIV nucleic acids in a urine sample obtained from said patient,

b) comparing the expression level of HIV nucleic acids to a predetermined value,

c) determining the renal function of said patient, and

d) determining from steps a) to c) whether the patient is afflicted with a HIVAN.

In a second aspect, the invention relates to a method for adjusting the anti-viral treatment and/or the immunosuppressive treatment administered to an HIV-infected patient or a HIV-infected kidney transplant recipient following its graft transplantation comprising the following steps of: (i) performing the method for diagnosing HIVAN above-described, and (ii) adjusting the anti-viral treatment and/or the immunosuppressive treatment.

In a third aspect, the invention relates to a kit for performing the method above-described, comprising at least a) means useful for measuring the expression level of HIV nucleic acids and b) means useful for determining the renal function in a patient.

In a fourth aspect, the invention relates to a method for diagnosing HIVAN in a HIV-infected kidney transplant recipient, comprising a step of determining the expression level of HIV nucleic acids in a kidney biopsy sample obtained from said recipient, wherein the presence of HIV nucleic acids in podocytes cells is indicative of HIVAN.

In a fifth aspect, the invention relates to method for distinguishing between HIVAN and non-HIVAN kidney disease in a HIV-infected patient, comprising a step of determining the expression level of HIV nucleic acids in a urine sample obtained from said patient, wherein the presence of HIV nucleic acids in said sample is indicative of HIVAN.

### DETAILED DESCRIPTION OF THE INVENTION

The invention is based, in part, on the discovery that following transplantation, HIV-1 is able to reinfect the kidney allograft despite undetectable viremia, which might influence the allograft prognosis (i.e. allograft survival rate). By virtue of development of a non-invasive diagnostic test of HIV-1 renal allograft infection using a quantitative PCR assay of HIV-1 RNA and HIV-1 DNA in urine, they have shown that only recipients with HIV-1 infection of the allograft had HIV-1 nucleic acids in urine. The inventors reported findings that HIV-1 infected kidney allografts in a cohort of patients with undetectable systemic HIV who received effective antiretroviral therapy. Of equal importance, they reported a new and noninvasive test for determining HIV-1 infection of the kidney allograft by measuring DNA and RNA levels in patients' urine. They further highlighted that unrecognized reinfection of the transplanted kidney by HIV-1 compromise allograft function, and is associated with the higher rejection rates observed in kidney transplant recipients with HIV.

In summary, they demonstrated reinfection of the kidney allograft with HIV-1 after transplantation in the HIV-infected recipient with well controlled (undetectable) viral disease. The identification of a noninvasive urine test to detect early reinfection that correlates with allograft HIV-1 infection facilitate the identification of donor and recipient factors associated with recurrent HIV renal disease.

Moreover, the inventors have shown that only detection of HIV nucleic acids combined with the renal function which may be determined for instance by the level of proteinuria in a urine sample obtained from a patient is useful for diagnosing HIV-associated nephropathy (HIVAN). Indeed, the level of proteinuria in the urine of patients with HIVAN is much higher than the level of proteinuria in the urine of HIV-positive patients with asymptomatical HIV-1 reinfection of the allograft.

Finally, the inventors have shown by in situ hybridization (ISH) the presence of HIV-1 RNA in podocytes or tubular cells, whereas HIV-1 RNA in plasma was undetectable and that only patients with HIV-1 RNA in podocytes are affected with HIVAN. Indeed, two distinct types of infections were observed, affecting either podocytes (5 of 13) or tubular cells (8 of 13). The former type of infection had a more severe clinical effect, resulting in nephrotic-range proteinuria and disease progression.

Thus, in a first aspect, the invention relates to a method for diagnosing HIV-associated nephropathy (HIVAN) in a HIV-infected patient, comprising the following steps of:
a) determining the expression level of HIV nucleic acids in a urine sample obtained from said patient,
b) comparing the expression level of HIV nucleic acids to a predetermined value,
c) determining the renal function of said patient, and
d) determining from steps a) to c) whether the patient is afflicted with a HIVAN.

In one embodiment, the invention relates to a method for diagnosing HIV-associated nephropathy (HIVAN) in a HIV-infected patient, comprising the following steps of:
a) determining the expression level of HIV nucleic acids in a urine sample obtained from said patient,
b) comparing the expression level of HIV nucleic acids to a predetermined value,
c) determining the renal function of said patient,
d) comparing the renal function to a predetermined value, and
e) determining from steps a) to d) whether the patient is afflicted with a HIVAN.

As used herein, the term “HIV” refers to the human immunodeficiency virus. HIV includes, without limitation, HIV-1. HIV may be either of the two known types of HIV, i.e., HIV-1 or HIV-2. The HIV-1 virus may represent any of the known major subtypes or clades (e.g., Classes A, B, C, D, E, F, G, J, and H) or outlying subtype (Group 0). Also encompassed are other HIV-1 subtypes or clades that may be isolated.

As used herein, the term “nucleic acids” refers to RNA or DNA molecules including single-stranded, double-stranded, oligonucleotide or polynucleotide. The term “nucleotide sequence” is used to refer to the ordering of nucleotides in an oligonucleotide or polynucleotide in a single-stranded form of nucleic acid.

As used herein, the term “determining” includes qualitative and/or quantitative detection (i.e. detecting and/or measuring the expression level) with or without reference to a control or a predetermined value. As used herein, “detecting” means determining if HIV nucleic acids are present or not in a biological sample and “measuring” means determining the amount of HIV nucleic acids in a biological sample. Typically the expression level of HIV nucleic acids can be determined by any method familiar to one of skill in the art. Such methods typically include the methods based on the detecting the HIV nucleic acids expression. HIV nucleic acids may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a HIV gene such as the HIV-1 gag, protease, p24, integrase and/or envelope nucleic acids. According to a first alternative, conditions for primer annealing may be chosen to ensure specific reverse transcription (where appropriate) and amplification; so that the appearance of an amplification product be a diagnostic of the presence of HIV nucleic acids. Otherwise, RNA may be reverse-transcribed and amplified, or DNA may be amplified, by hybridization with a suitable probe or any other appropriate method known in the art.

As used herein, the term “predetermined values” refers to the expression level of HIV nucleic acids and the renal function (determined by the level of proteinuria or by the estimated glomular filtration rate (eGFR)) in urine samples obtained from the general population or from a selected population of subjects. The predetermined reference value can be a threshold value or a range. For example, the selected population may be comprised individuals who have not previously had any sign or symptoms indicating the outcome of HIVAN such as of apparently healthy transplanted patient.

### Methods for Determining the Expression Level of HIV Nucleic Acids

Therefore, useful nucleic acid molecules, in particular oligonucleotide probes or primers, according to the invention include those which specifically hybridize with a HIV nucleic acids such as the HIV-1 gag, protease, p24, integrase and/or envelope nucleic acids.

As used herein, the terms “primer” and “probe” refer to the function of the oligonucleotide. A primer is typically extended by polymerase or ligation following hybridization to the target but a probe typically is not. A hybridized oligonucleotide may function as a probe if it is used to capture or detect a target sequence, and the same oligonucleotide may function as a primer when it is employed as a target binding sequence in an amplification primer. Degenerate primers and probes which specifically and sensitively amplify and allow detection of multiple clades of HIV-1 may be used.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. In contrast, low stringency hybridization conditions are those in which nucleic acids having a low degree of complementarity hybridize.

The terms “specific hybridization,” and “specifically hybridizes” refer to hybridization of a particular nucleic acid to a target nucleic acid without substantial hybridization to nucleic acids other than the target nucleic acid in a sample. Primers which specifically hybridize to target HIV RNA and/or DNA under stringent hybridization conditions and are specific for detection of HIV nucleic acids are well known in the art.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, complementarity to a specified second nucleotide sequence, indicating that 8 of 10 nucleotides of a sequence are complementary to the specified second nucleotide sequence.

Broadly described, a detection assay according to embodiments of the invention typically includes combining one or more sets of primers, dNTPs, a buffer, magnesium, a DNA polymerase, optionally a reverse transcriptase, and a sample to be assayed for presence of HIV nucleic acids in a reaction mixture.

A primer set included in a reaction mixture is any primer set described herein. In particular embodiments of the invention, more than one primer set is included in a reaction mixture. For example, two or more primers sets for use to detect HIV-1 protease, p24, integrase and/or envelope nucleic acids can be included in a reaction mixture.

In a further example, two or more primers sets for use to detect HIV-1 and HIV-2 can be included in a reaction mixture.

Magnesium can be included as a magnesium salt such as magnesium acetate, magnesium chloride or magnesium sulfate.

Any buffer compatible with the reagents and reaction can be used, illustratively including sodium phosphate buffer, potassium phosphate buffer, Tris-HCl buffer and Tricine buffer.

DNA polymerases include DNA polymerases derived from a strain of thermophilic microorganism. Preferred are DNA polymerases lacking a 5′ to 3′ exonuclease activity. Illustrative examples of DNA polymerases used in the present invention include Bacillus stearothermophilus, Bst, DNA polymerase; Thermus thermophilus, Tth, DNA polymerase; Thermus aquaticus, Taq, DNA polymerase; Thermococcus litoralis DNA polymerase; Pyrococcus furiosus, Pfu, DNA polymerase; and Bacillus caldotenax DNA polymerase.

Reverse transcriptase enzymes included in the reaction mixture illustratively include Moloney murine leukemia virus, MMLV, reverse transcriptase and avian myeloblastosis virus, AMV, reverse transcriptase.

A reaction mixture is then incubated at a temperature suitable for activity of the DNA polymerase and, where included, the reverse transcriptase. The temperature depends on the particular enzymes used and the nucleotide sequence of the desired target and can be determined by one of skill in the art without undue experimentation. The reaction mixture is incubated at the appropriate temperature for a time suitable for production of amplified nucleic acid. The reaction time will depend on the reaction conditions and can be determined by one of skill in the art without undue experimentation. In general, reaction time is in the range of about 15-60 minutes but can be longer or shorter depending on factors including the amount of template nucleic acid in the sample to be tested for presence of HIV nucleic acids.

In preferred embodiments, both a DNA polymerase and a reverse transcriptase are included in a reaction mixture. In a reaction mixture containing both a DNA polymerase and a reverse transcriptase, both DNA and RNA present in the sample are amplified allowing for robust production of amplified product as well as ease of use. In particular, a reaction mixture including both a DNA polymerase and a reverse transcriptase is preferred where a whole blood sample is used since both DNA and RNA of HIV are typically present.

Detection of amplified reaction products is achieved by any of various methods illustratively including detection of turbidity, fluorescence and/or electrophoresis pattern. In general, amplified reaction products produced in a reaction mixture containing a test sample, such as a sample obtained from a patient, is compared with any products produced in positive and/or negative controls.

Specific amplified reaction products may be detected instead of, or in addition to, detection of total amplified nucleic acid in the reaction product.

In a particular embodiment, a detectably labeled primer is included in a reaction mixture and a detectably labeled reaction product is produced. A signal from the detectably labeled reaction product is detected to determine whether amplified HIV nucleic acids are produced, indicative of presence of HIV nucleic acids in the sample tested. This method allows for detection of HIV specific reaction product absent detection of non-specific products in the reaction.

The terms “detectably labeled” and “detectable label” refers to a material detectable capable of producing a signal indicative of the presence of a detectably labeled nucleic acid by any appropriate method illustratively including spectroscopic, optical, photochemical, biochemical, enzymatic, electrical and/or immunochemical. Examples of detectable labels illustratively include a fluorescent moiety, a chemiluminescent moiety, a bioluminescent moiety, a magnetic particle, an enzyme, a substrate, a radioisotope and a chromophore. In a preferred embodiment, a detectable label is a fluorescent label.

In a particular embodiment, the quantitative HIV RNA viral load assays such as the COBAS® Amplicor HIV-1 Monitor assays (Roche Diagnostics) currently approved for in vitro diagnostic use in the United States is used for carrying out the invention. The test consists of independent steps for RNA isolation, reverse transcription and PCR (RT-PCR) amplification, and detection using a colorimetric readout. Viral RNA is released from the virions with guanidine isothiocyanate, and nucleic acid from the relatively impure lysate is precipitated with isopropanol. RT-PCR amplification occurs in a single tube using the thermostable recombinant enzyme Thermus thermophilus DNA polymerase (rTth pol), which has both RT and DNA polymerase activities. The PCR products are serially diluted and denatured, and single-stranded DNA is bound to microwells coated with HIV-specific oligonucleotide probes. An avidin-horseradish peroxidase (HRP) conjugate is added, binding to the biotin-labeled amplicon. The amount of bound amplicon is determined using an enzyme-linked immunosorbent assay (ELISA) plate reader after the addition of an HRP-specific colorimetric substrate.

### Methods for Determining the Renal Function

As used herein, the term “renal function” (or “kidney function”) in nephrology refers to an indication of the state of the kidney of a patient and its role in renal physiology.

In one embodiment, the renal function may be determined by determining the level of proteinuria in a urine sample obtained from said patient.

As used herein, the term “level of proteinuria” refers to any amount of protein passing through a podocyte that has suffered podocyte damage or through a podocyte mediated barrier that normally would not allow for any protein passage. In an in vivo system the term “proteinuria” refers to the presence of excessive amounts of serum protein in the urine. Proteinuria is a characteristic symptom of renal (kidney) diseases, nephrotic syndromes (i.e., proteinuria larger than 3.5 grams per day) and toxic lesions of kidneys.

For example, the presence of any amount of protein (typically albumin) in the urine sample, in combination with the presence of HIV nucleic acids, is indicative of HIVAN. For example, the amount of protein (typically albumin) in the urine can be greater than about 10 mg/dl (trace), or greater than about 30 mg/dl (1+), or greater than about 100 mg/dl (2+), or greater than about 300 mg/dl (3+), or greater about 1000 mg/dl (4+).

Methods of determining whether a patient has proteinuria are well known in the art and are routinely performed by medical professionals, for example using standard laboratory tests or using urine dipstick methods. Most proteinuria tests are based on the detection of albumin in the urine, and/or by determining the level of albumin in the urea as a function of the urine creatinine level, e.g. determining the urine albumin-to-creatinine ratio (UACR). Any proteinuria test can be used in conjunction with the methods of the present invention.

In one embodiment, it is determined that the patient is afflicted with HIVAN when the expression level of HIV nucleic acids and the level of proteinuria are higher than the predetermined values.

Typically, the predetermined values correspond to an absence of expression of HIV nucleic acids (inferior to 50 copies/ml) and to an absence of proteniuria (inferior to 300 mg/1). Accordingly, a patient with a level of proteinuria higher than 300 mg/l in combination with the presence of HIV nucleic acids, is indicative of HIVAN.

Alternatively, the renal function may be measured by other means, such as blood urea nitrogen, glomular filtration rate, inulin assay, or estimated glomular filtration rate (eGFR). Kidney function may be at 100, 95, 90, 85, 80, 70, 60, 50, 40, 30, 20, 10%, or less, of normal levels as measured by any of these assays. A normal range for eGFR is 60 to 90 ml/min. Accordingly, in some embodiments, a patient with a decreased renal function may exhibit an eGFR of less than 60, 55, 50, 45, 40, 35, 30 ml/min.

Typically, the renal function may be determined by calculating the glomerular filtration rate estimated by the MDRD formula (eGFR) (based on the serum creatinine level as described in the Example below).

In one embodiment, it is determined that the patient is afflicted with HIVAN when the expression level of HIV nucleic acids is higher than the predetermined value and the renal function is lower than the predetermined value.

Typically, the predetermined value may be 60 ml/min. Accordingly, a patient with an estimated glomerular filtration rate (eGFR) lower than 60 ml/min in combination with the presence of HIV nucleic acids, is indicative of HIVAN.

In one embodiment, the renal function may be determined by determining the level of proteinuria in a urine sample obtained from said patient and also by determining blood urea nitrogen, glomular filtration rate, inulin assay, or estimated glomular filtration rate (eGFR).

In one embodiment, the HIV-infected patient is a kidney transplant recipient.

In a second aspect, the invention relates to a method for adjusting the anti-viral treatment and/or the immunosuppressive treatment administered to an HIV-infected patient or a HIV-infected kidney transplant recipient following its graft transplantation comprising the following steps of: (i) performing the method for diagnosing HIVAN of the invention, and (ii) adjusting the anti-viral treatment and/or the immunosuppressive treatment.

In one embodiment, the anti-viral treatment (e.g. an antiretroviral therapy as described below) is administered at an increased dose.

In another embodiment, the immunosuppressive treatment is administered at a decreased dose.

In still another embodiment, the anti-viral treatment is administered at an increased dose and the immunosuppressive treatment is administered at a decreased dose.

Several classes of immunosuppressive drugs may be used alone or in combination in HIV-infected kidney transplant recipients.

For instance, a combination of steroids, a calcineurin inhibitor (tacrolimus—Prograf®) and mycofenolate mofetil (Cellecpt®) may be used for induction therapy. Additionally an interleukin-2 receptor inhibitor (basiliximab—Simulect®) or a lymphocyte depleting agent (Thymoglobulin®) may also be used for induction therapy.

The invention also provides a method for determining whether an HIV-infected patient or a HIV-infected kidney transplant recipient following its graft transplantation is a candidate for treatment with highly active antiretroviral therapy (HAART) comprising a step of performing the method for diagnosing HIVAN of the invention.

In some embodiments, such methods further comprise a subsequent step of administering highly active antiretroviral therapy (HAART) to the patient in need thereof.

Several classes of antiretroviral drugs are used in antiretroviral therapy and HAART. There are 5 classes of antiretrovirals which are specified hereinafter.

Nucleotide reverse transcriptase inhibitors (nRTIs) competitively inhibit the HIV reverse transcriptase enzyme. Contray to NRTIs, they do not require initial phosphorylation. Non-limiting examples include Truvada® (Tenofovir and Emtricabine) and Viread® (Tenofovir)

Nucleoside reverse transcriptase inhibitors (NRTIs) are phosphorylated to active metabolites that compete for incorporation into viral DNA. They inhibit the HIV reverse transcriptase enzyme competitively and terminate synthesis of DNA chains. Non-limiting examples include Viramune® (Neviparin), Kivexa® (Abacavir and Lamivudine), Combivir® (Idovudine and Lamivudine), Retrovir® (Zidovudine), Videx® (DDI), Epivir® (Lamivudine), Ziagenavir® (Abacavir).

Non-nucleoside reverse transcriptase inhibitors (NNRTIs) bind directly to the reverse transcriptase enzyme. Non-limiting examples include Sustiva® (Efavirenz), Emtriva® (Emtricabine).

Protease inhibitors (PIs) inhibit the viral protease enzyme that is crucial to maturation of immature HIV virions after they bud from host cells. Non-limiting examples include Crixivan® (Indinavir), Kaletra® (Lopinavir and Ritonavir), Reyataz® (Atzanavir), Telzir® (Fosanprenavir), Invirase® (Saquinavir), Prezistza® (Duranavir).

Further known are entry inhibitors (EIs), sometimes called fusion inhibitors, they interfere with the binding. Non-limiting examples include Fuzeon® (Enfuvirtide).

Integrase inhibitors prevent HIV DNA from being integrated into human DNA. Non-limiting examples include Insentress® (Raltegravir).

The usual HAART regimen combines three or more different drugs such as two nucleoside reverse transcriptase inhibitors (NRTIs) and a protease inhibitor (PI), two NRTIs and a non-nucleoside reverse transcriptase inhibitor (NNRTI) or other such combinations.

The invention further relates to a method for distinguishing between HIVAN and non-HIVAN kidney disease in a HIV-infected patient, comprising a step of determining the expression level of HIV nucleic acids in a urine sample obtained from said patient, wherein the presence of HIV nucleic acids in said sample is indicative of HIVAN.

In a third aspect, the invention relates to a kit for performing the methods of the invention, comprising at least a) means useful for measuring the expression level of HIV nucleic acids and b) means useful for determining the renal function in a patient.

In one embodiment, means useful determining the renal function are any means for determining the level of proteinuria in a urine sample obtained from a patient

In another embodiment, means useful determining the renal function of a patient are any means for determining or calculating blood urea nitrogen, glomular filtration rate, inulin assay, or estimated glomular filtration rate (eGFR).

In one particular embodiment, the kit for performing the methods of the invention, comprising at least a) means useful for measuring the expression level of HIV nucleic acids and b) means useful for determining the renal function selected from the group consisting of means for determining or calculating blood urea nitrogen, glomular filtration rate, inulin assay, or estimated glomular filtration rate (eGFR) and means useful for determining the level of proteinuria in a urine sample of a patient.

In a fourth aspect, the invention relates to a method for diagnosing HIVAN in a HIV-infected kidney transplant recipient, comprising a step of determining the expression level of HIV nucleic acids in a kidney biopsy sample obtained from said recipient, wherein the presence of HIV nucleic acids in podocytes cells is indicative of HIVAN.

As previously described, for carrying out the invention, the level of expression of HIV nucleic acids is measured by measuring RNA expression (transcription products).

This measurement can be performed by various methods which method which are well known to the person skilled in the art, including in particular quantitative methods involving reverse transcriptase PCR (RT-PCR), such as real-time quantitative RT-PCR (qRT-PCR), and methods involving the use of DNA arrays (macroarrays or microarrays) and in situ hybridizations.

The invention will be further illustrated by the following figures and examples. However, these examples and figures should not be interpreted in any way as limiting the scope of the present invention.

### FIGURES

FIG. 1: HIV-1 reinfects podocytes after transplantation and negatively impacts kidney function. Representative graphics of estimated glomerular filtration rate (eGFR) (circle plot) and albuminuria (square plot) for each patient. The first two patients were treated with plasmapheresis (inverted triangle) and high dose cyclosporine (rectangle). Blue arrows represent transplant biopsies.

FIG. 2: Functional impact of tubular cells reinfection by HIV-1. Representative graphics of estimated glomerular filtration rate (eGFR) (circle plot) and albuminuria (square plot) for each patient. Arrows represent transplant biopsies; cones represent steroids pulses for acute cellular rejection.

### EXAMPLE

### Material & Methods

Patients and Design of the Study:

This study was performed in the Department of Kidney Transplantation of the Necker Hospital from Jun. 1, 2006 to Oct. 31, 2011. Patients with HIV type 1 (HIV-1) infection were eligible for transplantation if, prior to transplantation, they had a CD4+T-cell count of at least 200 cells per cubic millimeter, undetectable plasma HIV RNA levels (<50 copies per milliliter), and they were stable on highly active antiretroviral therapy (HAART) for at least 6 months. CDC C stage patients have also been included in the program with the exception of those with a previous history of progressive multifocal leukoencephalopathy, primary central nervous system lymphoma and visceral Kaposi's sarcoma.

Immunosuppressive regimen was similar for all patients and included steroids, tacrolimus (Prograf®, Astellas) and mycopheno late mofetil (Cellcept®, Roche Pharmaceuticals). Induction therapy was performed with the anti-IL-2 receptor antibody basiliximab in all cases (Simulect®, Novartis Pharma AG), except in patients with preformed donor specific antibodies (DSA), in whom induction therapy was changed for rabbit antithymocyte globulin (Thymoglobulin®, Genzyme). In the particular case of sensitized patients, intravenous immunoglobulins were added. Tacrolimus trough levels were carefully monitored due to pharmacokinetic drug interactions.

Anti-retroviral combination therapy was similar for all patients and consisted of two nucleoside analog reverse-transcriptase inhibitors and one protease inhibitor. Drug doses were adjusted for kidney function, with frequent adjustments being required particularly in the early post-transplantation period and during periods of graft dysfunction. All patients continued their HAART regimen after transplantation. All patients with a functioning allograft had protocol biopsies at 3 and 12 months post-transplantation.

Renal Function:

The serum creatinine level was measured monthly during the first year and 3-monthly thereafter using a Synchron Cx4 autoanalyzer (Beckman Coulter, Villepinte, France). Glomerular filtration rate was estimated by the MDRD formula (eGFR) {Stevens et al., 2009}.

Biopsy Samples and Morphological Analysis:

Transplant kidney biopsies were fixed in alcohol-formalin-acetic acid solution and embedded in paraffin. Four-μm sections were stained with the Periodic Acid Schiff (PAS), Masson's trichrome and hematoxylin and eosin (H&E). Each biopsy was interpreted in accordance with the BANFF 2007 criteria. Electron microscopy analyses were performed as previously described {Mollet et al., 2009}.

Urine Collection and HIV-1 RNA/DNA Testing:

Urines were longitudinally collected for each patient, at 3 and 12 months post transplantation and when a biopsy for cause was performed. Fifty mL of freshly collected urine samples were immediately centrifuged at 1000 g for 10 minutes at 4° C. RNA was extracted from 3.2 mL supernatant with Qiagen kit and HIV-1 RNA was quantified with Biocentric ultrasensitive assay. Pellets were used for DNA extraction with QIAamp DNA mini kit (QIAGEN, Courtaboeuf, France), according to the manufacturer's instructions. HIV-1 DNA was then quantified using an ultrasensitive assay from Biocentric {Avettand-Fenoel et al., 2009; Avettand-Fenoel et al., 2008}.

Plasma HIV-1 RNA and Blood Cell Associated HIV-1 DNA:

Plasma HIV-1 RNA was quantified as previously described using the COBAS® AmpliPrep/COBAS® TaqMan® HIV-1 Test, v2.0, (Roche Diagnostics, Meylan, France) RT PCR assay, with a detection limit of 20 copies/mL. Cell associated HIV-1 DNA was quantified on whole blood samples using the real-time HIV-1 DNA assay {Avettand-Fenoel et al., 2009} (Biocentric, Bandol, France) with a detection limit of 1.7 log copies/10⁶ cells. Plasma HIV-1 RNA levels were monitored at day 0, day 7, and then monthly until 6-months post transplantation. Thereafter, HIV-1 RNA levels were monitored every 3 months.

HIV-1 DNA Detection on Frozen Biopsies:

Total DNA was extracted from renal tissues using QIAamp tissue kit (QIAGEN, Courtaboeuf, France), according to the manufacturer's instructions. Cell associated HIV-1 DNA was quantified using the real-time HIV-1 DNA assay {Avettand-Fenoel et al., 2009} (Biocentric, Bandol, France).

In Situ Hybridization:

Alcohol-formalin-acetic acid solution-fixed, paraffin-embedded tissues were assayed for HIV-1 RNA expression using a previously described digoxygenin-anti-digoxygenin technique {Hirsch et al., 1995}. The digoxigenin-UTP-labeled riboprobe used spans the whole genome of HIV-1 (Lofstrand Labs Ltd, Gaithersburg, Md., USA). For localization of infected cells in the tissues, nitroblue tetrazolium-5-bromo-4-chloro-3-indollylphosphate toluidinium (NBT-BCIP) revelation was used. The specificity of the hybridization signal was systematically checked by hybridizing sense probes on parallel sections and anti-sense probes on uninfected renal tissues. ISH-stained tissues were visualized and photographed with a Olympus Proxis microscope and a Zeiss Axio Cam ICc1.

Statistical Analysis:

Comparisons between groups were performed using conventional statistics for matched data, including the Mann-Whitney test. Probability values <0.05 were considered statistically significant. Analyses were performed with GraphPad Prism 5 (GraphPad software, La Jolla, Calif.).

Results

HIV-1 Reinfects the Kidney Allograft:

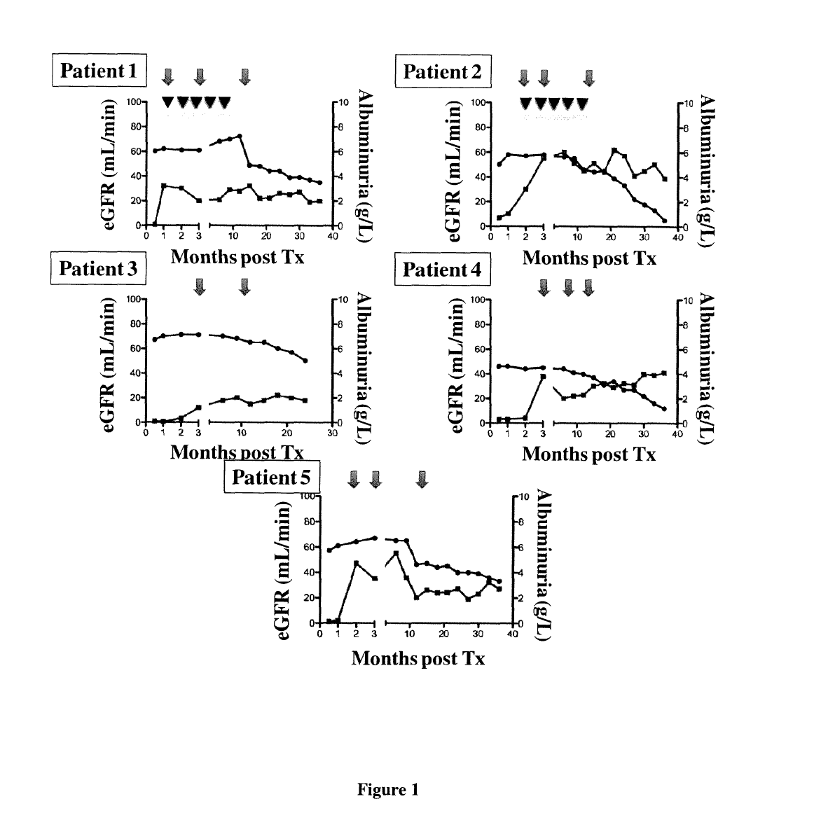

During the 2006-2011 period, 939 kidney transplantations (Tx) were performed in our center. Among them, nineteen HIV-1 infected patients were engrafted. After Tx, five (26.3%) of the nineteen patients developed nephrotic range proteinuria from day-15 to month-3 post Tx (FIG. 1). Demographic characteristics of these patients are presented in Table 1. To understand the etiology of proteinuria, we performed a transplant biopsy in each case. Surprisingly, all five biopsies displayed normal histology on light microscopy with negative immunofluorescence. Foot process effacement was observed using electron microscopy. This led us to consider the diagnosis of focal and segmental (FSGS) recurrence for the first two patients, in spite of a fairly established native kidney histological diagnosis of HIVAN. In these two cases, we began our current therapeutic protocol for recurrent FSGS {Canaud et al., 2009}, consisting in plasmapheresis, steroids and high dose of cyclosporine. Unfortunately, this treatment failed to improve proteinuria (FIG. 1). Subsequent biopsies performed at 3- and 12-months post-Tx in all five patients revealed the progressive constitution of FSGS lesions in all cases. These lesions were associated with a progressive decline of renal function (FIG. 1).

The occurrence of unexpected and early FSGS lesions, resistant to usual therapy, led us to hypothesize that HIV-1 could infect the allograft despite undetectable plasma HIV-1 RNA throughout follow-up. To test our hypothesis, we first performed PCR for HIV-1 DNA on indication biopsies and protocol biopsies performed at 3- and 12-months post Tx. Surprisingly, we observed that all five patients had detectable HIV-1 DNA on early indication biopsies as well as on protocol biopsies performed at 3- and 12-months post Tx. In order to investigate whether the presence of HIV-1 DNA on allograft biopsies could be caused by infiltrating inflammatory cells or due to a true reinfection of kidney cells, in situ hybridization (ISH) for HIV-1 RNA was performed on the kidney biopsies. ISH revealed that HIV-1 RNA was detectable in all biopsies with HIV-1 DNA positive PCR. Importantly, we could detect HIV RNA in glomeruli and in very few tubular cells in these 5 patients. Positive cells were outlying glomerular cells with typical morphologic features of podocytes.

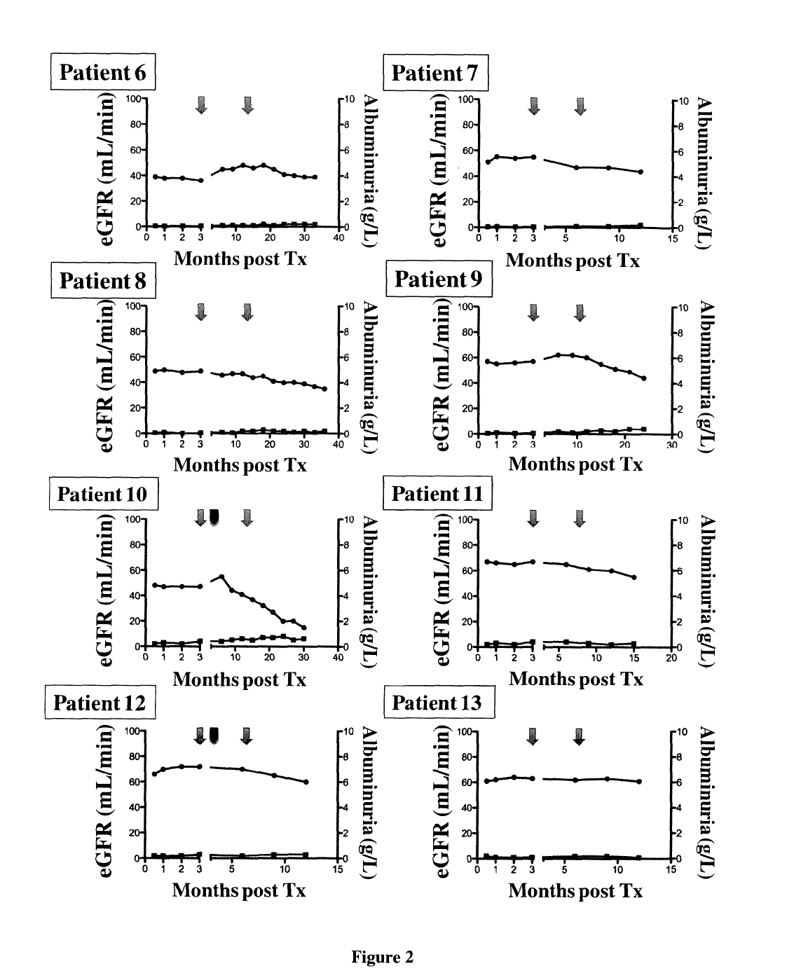

We therefore concluded that HIV-1 is able to infect the kidney allograft even with no detectable plasma HIV-1 RNA levels. We next evaluated the rest of our cohort of HIV-1-infected transplanted patients to investigate whether HIV-1 could also be detected in the allograft in the absence of proteinuria. Demographical characteristics of the 14 remaining patients are presented in Table 2 and FIG. 2. We performed HIV-1 DNA PCR on all biopsies (3 and 12 months post Tx and indication biopsies). Interestingly, among the fourteen remaining patients, eight had renal positive HIV-1 DNA (patients 6 to 13). In those cases, all biopsies were positive at 3 months and remained positive at 12 months post Tx. ISH revealed that six patients among eight had positive HIV-1 RNA in tubular cells and some vascular sections. Of note, in two patients, electron microscopy revealed the presence of tubuloreticular inclusions in endothelial cells. These inclusions consist of structures located within dilated cisternae of endoplasmic reticulum are frequently, but not specifically, observed during HIVAN {Strauss et al., 1989}.

Importantly, the two remaining patients with a positive HIV-1 DNA PCR in the allograft (Patient 7 and 9, Table 2) had positive interstitial ISH at 3 months post Tx with an aspect of acute cellular rejection. In this particular setting, tubular and infiltrating cells were undistinguishable. Of interest, at 12 months post Tx, despite treatment with high dose of steroids, biopsies showed for both patients signs of acute cellular rejection and the persistence of positive ISH in both infiltrating and tubular cells. Plasma HIV-1 RNA was, and remained, undetectable in all patients during follow-up.

Predictive urinary test for infection: Considering the high incidence of allograft infection and the difficulty of performing routine ISH, the development of an accurate non-invasive diagnostic test of HIV-1 renal allograft infection would be of considerable value. To this end, we developed a quantitative PCR assay for the detection of HIV-1 RNA and HIV-1 DNA in the urine. We prospectively collected urine samples from patients just prior to protocol (3 and 12 months post Tx) and biopsies for causes. After centrifugation of fresh urine, HIV-1 RNA was extracted from supernatant and pellets were used for DNA extraction Sequentially collected urine samples showed that HIV-1 RNA and/or HIV-1 DNA were detectable only in the group of patients with a HIV-infected allograft. Importantly, HIV-1 RNA and/or HIV-1 DNA presence in urine was strictly associated with the presence of positive ISH in biopsies (Table 5). Importantly, HIV-1 RNA and DNA were detectable at 3 months post Tx and remained persistent at 12 months. Finally, to exclude any positivity due to infiltrating leucocytes, we performed HE staining, CD3 and CD68 immunostaining on cells isolated from the pellet and observed that no inflammatory cells were present.

TABLE 1
Demographic characteristics of HIV-1 kidney transplant recipients with podocyte reinfection.

| | | | | | | | Duration of | | | | | | |
|---|---|---|---|---|---|---|---|---|---|---|---|---|---|
| | | | | Nadir | CD4+ | HIV-1 DNA | undetectable | | | | | | |
| | | | | CD4+ | count | per PBMC at | plasma | | | | | | |
| | | | Cause | count | per | Day 0 | HIV-1 RNA | | Age at | Duration | | | |
| | | Geographic | of | per | mm3 at | (log copies/ | before Tx | IDS | HD | of HD | HCV | | |
| Patient | Sex | origin | ESRD | mm3 | Day 0 | 10⁶ cells) | (year) | stage | (year) | (year) | status | Induction | Donor |
| 1 | M | Caucasian | MPGN | 155 | 359 | 2.9 | 3.5 | No | 53 | 2 | Negative | Basilix. | DC |
| 2 | M | Caucasian | IgAN | 453 | 1216 | 2.4 | 1.7 | No | 36 | 2 | Negative | Basilix. | DC |
| 3 | F | Sub Saharan | HIVAN | 245 | 906 | 2.5 | 8.3 | No | 30 | 11 | Negative | Basilix. | DC |
| | | Afr. | | | | | | | | | | | |
| 4 | M | Sub Saharan | HIVAN | 340 | 575 | <1.7 | 2.4 | No | 50 | 3 | Positive | Basilix. | DC |
| | | Afr. | | | | | | | | | | | |
| 5 | F | Sub Saharan | HIVAN | 107 | 382 | 3.6 | 8.0 | No | 32 | 7 | Negative | Basilix. | LR |
| | | Afr. | | | | | | | | | | | |

Sub Saharan Afr.: Sub Saharan African;
ESRD: End Stage Renal Disease;
MPGN: Membranoproliferative Glomerulonephritis;
HIVAN: HIV Associated Nephropathy;
ADPKD: Autosomal Polycystic Kidney Disease;
IgAN: IgA Nephropathy;
PBMC: Peripheral Blood Mononuclear Cell;
HD: Hemodialysis,
HCV: Hepatitis C Virus;
Basilix.: Basiliximab;
DC: Deceased Donor;
LR: Living Related Donor;
Tx: Transplantation.

TABLE 2
Demographic characteristics of HIV-1 kidney transplant recipients without nephrotic range proteinuria

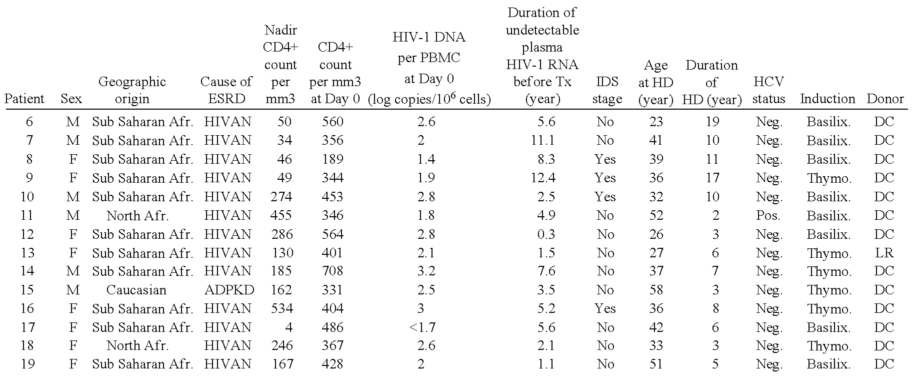

From Patient 6 to 13 (grey area): HIV-1 reinfection of kidney cells, from Patient 14 to 19: no kidney HIV-1 reinfection.
Sub Saharan Afr.: Sub Saharan African;
North Afr.: North African;
HIVAN: HIV Associated Nephropathy;
ADPKD: Autosomal Polycystic Kidney Disease;
ESRD: End Stage Renal Diasease;
PBMC: Peripheral Blood Mononuclear Cell;
HD: Hemodialysis, HCV: Hepatitis C Virus;
Neg.: Negative;
Pos.: Positive;
Basilix.: Basiliximab;
Thymo.: Antithymocyte Globulins;
DC: Deceased Donor;
Tx: Transplatation.

TABLE 5
Urinary detection of HIV-1 RNA and/or DNA at 3 and
12 months post transplantation.

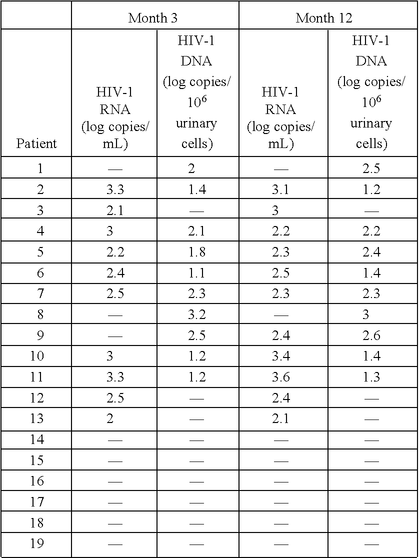

Strong grey area: patients with podocyte reinfection; Soft grey area: patients withtubular cells reinfection.

### DISCUSSION

Here we demonstrate that HIV-1 can reinfect kidney epithelial cells after transplantation, even though plasma HIV-1 RNA is undetectable. Importantly, we describe two different forms of allograft infection. In the first case, podocytes are the main targets of HIV-1, and infection is associated with nephrotic range proteinuria, progressive development of FSGS and poor transplant outcome. In the second case, HIV-1 can infect tubular cells from the kidney allograft with fewer clinical manifestations. Moreover, we provide an easy and new tool to non-invasively assess transplant kidney cell reinfection by the virus.

Infection of the kidney allograft while viral replication is undetectable in plasma was an unexpected finding. Indeed, all of these HIV-1 patients were registered on the transplant list as patients with controlled disease and thus were expected to benefit from kidney transplantation. How the virus reinfects kidney cells soon after kidney transplantation is an unsolved issue as repeated blood samples failed to detect viremia. One hypothesis is the ‘blips’ phenomenon, where HIV-1 patients receiving efficient HAART still experience intermittent episodes of detectable viremia {Nettles et al., 2005}, or very low-level replication under PCR detection limit. A second hypothesis is the direct transfer of HIV-1 RNA from recipient infected T-cells to donor kidney cells, as has been recently shown in vitro {Chen et al., 2011}. Furthermore, it remains unclear how HIV-1 enters podocytes and tubular cells as no HIV-1 receptors have as yet been described in the kidney {Wyatt et al., 2012}. Once in kidney cells, the virus is known to remain transcriptionally active in patients with undetectable blood plasma viremia under HAART. Finally, allograft reinfection can be interpreted as the consequence of immunosuppressive drug and/or insufficient HAART with suboptimal antiretroviral diffusion in the graft. We were unable to establish the particular factor (s) that either favour allograft reinfection or determine the surprising dichotomy of nephron segment reinfection (i.e. podocytes vs. tubular cells).

We provide evidence that transplant reinfection by HIV-1, at least in podocytes, shortens allograft survival rate. In native kidneys, HIV-1 infection of podocytes leads to cell dysfunction and to the development of a collapsing phenotype, specifically in the Afro-American population. This phenotype has been associated to G1 and/or G2 APOL1 variants. Caucasians, who do not express the G1 and/or G2 APOL1 variants, display various forms of glomerular lesions with immune deposits due to HIV-1, but usually have no features of HIVAN. Here, we observed only classical forms of FSGS without any immune deposits. We can formulate at least two non-mutually exclusive hypotheses to explain these lesions. First, it might be related to the low rate of G1 and/or G2 variants of APOL1 observed in the donors in this cohort, supposing that only donor variant is of importance regarding the high rate of G1 and/or G2 APOL1 variant in recipients. Secondly, immunosuppressive regimen such as steroids may limit immune deposits.

In our cohort, tubular reinfection seems to have less functional impact but the follow-up is certainly too short. We cannot exclude that tubular cell infection contributes to the shortened allograft survival observed in previous reports. A high rate of acute cellular rejection has been described in renal allografts of this population. Here, we observed that the two patients who developed what we called “acute cellular rejection”, revealed on ISH the presence of HIV-1 RNA either in infiltrating leukocytes or in adjacent tubular cells. Persisting virus in infiltrating inflammatory cells (whereas plasma HIV-1 RNA is undetectable) has been previously described {Zhang et al., 1999}, but the infiltration of inflammatory cells could be the result of an immune reaction against tubular cells infected by HIV-1.

In order to facilitate the monitoring of transplant infection with HIV-1, we developed a sensitive urinary assay. The detection of HIV-1 DNA and HIV-1 RNA in urine samples seems to be the consequence of kidney cell infection. Indeed, urinary HIV-1 DNA and/or HIV-1 RNA were exclusively detected in patients with positive RNA ISH either in podocytes or in tubular cells. Furthermore, light microscopic examination and immunocytochemistry of urine pellet exclude that HIV-1 DNA and/or HIV-1 RNA derived from infiltrating inflammatory cells. We cannot formally exclude that HIV-1 could be filtered from the plasma into the urine, even in the absence of detectable viremia, but its detection only in the setting of a positive HIS in the graft supports our hypothesis.

In conclusion, our work revealed the capacity of HIV-1 to infect the kidney allograft despite undetectable viremia. Urine testing appears as a promising non-invasive method of diagnosing HIV-1 reinfection, although it remains to be confirmed in a larger cohort. Finally, our data strongly support the need for close proteinuria monitoring in assessing the outcome of HIV-infected kidney transplant recipients.

### REFERENCES

Throughout this application, various references describe the state of the art to which this invention pertains. The disclosures of these references are hereby incorporated by reference into the present disclosure.
Adih, W. K., Selik, R. M. & Hu, X. Trends in Diseases Reported on US Death Certificates That Mentioned HIV Infection, 1996-2006. J Int Assoc Physicians AIDS Care (Chic) 10, 5-11 (2011).
Avettand-Fenoel, V., et al. HIV-DNA in rectal cells is well correlated with HIV-DNA in blood in different groups of patients, including long-term non-progressors. AIDS (London, England) 22, 1880-1882 (2008).
Avettand-Fenoel, V., et al. LTR real-time PCR for HIV-1 DNA quantitation in blood cells for early diagnosis in infants born to seropositive mothers treated in HAART area (ANRS CO 01). Journal of medical virology 81, 217-223 (2009).
Canaud, G., et al. Intensive and prolonged treatment of focal and segmental glomerulosclerosis recurrence in adult kidney transplant recipients: a pilot study. Am J Transplant 9, 1081-1086 (2009).
Chen, P., et al. Virological synapses allow HIV-1 uptake and gene expression in renal tubular epithelial cells. J Am Soc Nephrol 22, 496-507 (2011).
Cohen, M. S., Shaw, G. M., McMichael, A. J. & Haynes, B. F. Acute HIV-1 Infection. The New England journal of medicine 364, 1943-1954 (2011).
Hirsch, V. M., et al. Induction of AIDS by simian immunodeficiency virus from an African green monkey: species-specific variation in pathogenicity correlates with the extent of in vivo replication. Journal of virology 69, 955-967 (1995).
Kumar, M. S., et al. Safety and success of kidney transplantation and concomitant immunosuppression in HIV-positive patients. Kidney international 67, 1622-1629 (2005).
Marras, D., et al. Replication and compartmentalization of HIV-1 in kidneyepithelium of patients with HIV-associated nephropathy. Nature medicine 8, 522-526 (2002).
Mollet, G., et al. Podocin inactivation in mature kidneys causes focal segmental glomerulosclerosis and nephrotic syndrome. J Am Soc Nephrol 20, 2181-2189 (2009).
Nettles, R. E., et al. Intermittent HIV-1 viremia (Blips) and drug resistance in patients receiving HAART. JAMA: the journal of the American Medical Association 293, 817-829 (2005).
Solid organ transplantation in the HIV-infected patient. Am J Transplant 4 Suppl 10, 83-88 (2004).
Stevens, L. A. & Levey, A. S. Measured GFR as a confirmatory test for estimated GFR. J Am Soc Nephrol 20, 2305-2313 (2009).
Stock, P. G., et al. Outcomes of kidney transplantation in HIV-infected recipients. The New England journal of medicine 363, 2004-2014 (2010).
Strauss, J., et al. Renal disease in children with the acquired immunodeficiency syndrome. The New England journal of medicine 321, 625-630 (1989).
Touzot, M., et al. Renal transplantation in HIV-infected patients: the Paris experience. Am J Transplant 10, 2263-2269 (2010).
Wyatt, C. M., Meliambro, K. & Klotman, P. E. Recent progress in HIV-associated nephropathy. Annual review of medicine 63, 147-159 (2012).
Zhang, L., et al. Quantifying residual HIV-1 replication in patients receiving combination antiretroviral therapy. The New England journal of medicine 340, 1605-1613 (1999).

1. A method for diagnosing HIV-associated nephropathy (HIVAN) in a HIV-infected patient, comprising the following steps of:
a) determining the expression level of HIV nucleic acids in a urine sample obtained from said patient,
b) comparing the expression level of HIV nucleic acids to a predetermined value,
c) determining the renal function of said patient, and
d) determining from steps a) to c) whether the patient is afflicted with a HIVAN.

2. The method according to claim 1, wherein the renal function is determined by determining the level of proteinuria in a urine sample obtained from said patient.

3. The method according to claim 2, wherein it is determined that the patient is afflicted with HIVAN when the expression level of HIV nucleic acids and the level of proteinuria are higher than the predetermined values.

4. The method according to claim 3, wherein the predetermined values correspond to an absence of expression of HIV nucleic acids (inferior to 50 copies/ml) and to an absence of proteniuria (inferior to 300 mg/1).

5. The method according to claim 2, wherein an expression level of HIV nucleic acids superior to 50 copies/ml in combination with a level of proteinuria higher than 300 mg/l is indicative for said patient to be afflicted with HIVAN.

6. The method according to claim 1, wherein the renal function is determined by calculating the estimated glomular filtration rate (eGFR).

7. The method according to claim 6, wherein the expression level of HIV nucleic acids is higher than the predetermined value and the renal function is lower than the predetermined value.

8. The method according to claim 7, wherein the predetermined value for the expression level of HIV nucleic acids corresponds to an absence of expression of HIV nucleic acids (inferior to 50 copies/ml) and the predetermined value for the renal function corresponds to an eGFR equal to 60 ml/min.

9. The method according to claim 7, wherein an expression level of HIV nucleic acids superior to 50 copies/ml in combination with an eGFR lower than 60 ml/min is indicative for said patient to be afflicted with HIVAN.

10. The method according to claim 1, wherein the HIV-infected patient is a kidney transplant recipient.

11. A method for adjusting the anti-viral treatment and/or the immunosuppressive treatment administered to an HIV-infected patient or a HIV-infected kidney transplant recipient following its graft transplantation comprising the following steps of: (i) performing the method for diagnosing HIVAN according to claim 1, and (ii) adjusting the anti-viral treatment and/or the immunosuppressive treatment.

12. A method for preventing or treating HIVAN in a patient in need thereof comprising diagnosing HIV-associated nephropathy (HIVAN) in a HIV-infected patient, comprising the steps of:
a) determining the expression level of HIV nucleic acids in a urine sample obtained from said patient,
b) comparing the expression level of HIV nucleic acids to a predetermined value,
c) determining the renal function of said patient, and
d) determining from steps a) to c) whether the patient is afflicted with a HIVAN, and based on said determination, providing an-increased dose of an anti-viral drug and/or a decreased dose of an immunosuppressive drug to said patient.

13. A method for determining whether an HIV-infected patient or a HIV-infected kidney transplant recipient following its graft transplantation is a candidate for treatment with highly active antiretroviral therapy (HAART) comprising a step of performing the method for diagnosing HIVAN according to claim 1.

14. A kit for performing the method according to claim 1, comprising at least a) means useful for measuring the expression level of HIV nucleic acids and b) means useful for determining the renal function in a patient.

15. A method for diagnosing HIV-associated nephropathy (HIVAN) in a HIV-infected patient, comprising:
performing the steps of:
a) determining the expression level of HIV nucleic acids in a urine sample obtained from said patient and determining the renal function of said patient using a kit comprising at least i) means useful for measuring the expression level of HIV nucleic acids and ii) means useful for determining the renal function in a patient, and
b) determining from said expression level of HIV nucleic acids and said renal function whether the patient is afflicted with a HIVAN.

16. A method for diagnosing HIVAN in a HIV-infected kidney transplant recipient, comprising a step of determining the expression level of HIV nucleic acids in a kidney biopsy sample obtained from said recipient, wherein the presence of HIV nucleic acids in podocytes cells is indicative of HIVAN.

17. A method for distinguishing between HIVAN and non-HIVAN kidney disease in a HIV-infected patient, comprising a step of determining the expression level of HIV nucleic acids in a urine sample obtained from said patient, wherein the presence of HIV nucleic acids in said sample is indicative of HIVAN.
